(19) United States
(12) Patent Application Publication
(10) Pub. No.: US 2020/0015729 A1
(43) Pub. Date: Jan. 16, 2020

(54) METHOD FOR DETERMINING THE STRESS LEVEL OF AN INDIVIDUAL

(71) Applicants: Commissariat A L'Energie Atomique et aux Energies Alternatives, Paris (FR); Universite Grenoble Alpes, Saint Martin D'Heres (FR)

(72) Inventors: Gael VILA, Grenoble Cedex 09 (FR); Christelle GODIN, Grenoble Cedex 09 (FR); Aurelie CAMPAGNE, Fontaine (FR); Sylvie CHARBONNIER, Echirolles (FR)

(73) Assignees: Commissariat A L'Energie Atomique et aux Energies Alternatives, Paris (FR); Universite Grenoble Alpes, Saint Martin D'Heres (FR)

(21) Appl. No.: 16/509,706

(22) Filed: Jul. 12, 2019

(30) Foreign Application Priority Data
Jul. 13, 2018 (FR) 18 56504

(51) Int. Cl. A61B 5/16; A61B 5/00; G06F 17/18

(52) U.S. Cl. CPC A61B 5/165; G06F 17/18; A61B 5/7225; A61B 5/7275

(57) ABSTRACT

The invention is a method for determining a stress level of an individual, depending on a physiological parameter of the individual, the method comprising:
a) measuring a physiological-parameter value in various calibration periods during a calibration time period;
b) defining a range of variation in the physiological-parameter values measured in the various calibration periods;
c) depending on the range of variation, establishing a membership function defining a stress level of the individual depending on the physiological-parameter value, the stress level defined by the membership function being able to vary between:
   a rest level, corresponding to a rest state of the individual;
   a stressed level, corresponding to a stressed state of the individual;
   and at least one intermediate level, comprised between the rest level and the stressed level;
d) following c), measuring a physiological-parameter value during a measurement period, and determining the stress level of the individual by applying the membership function to the measured parameter value.

### TECHNICAL FIELD

The technical field of the invention is the determination of a stress level of an individual on the basis of at least one physiological-parameter measurement carried out on the individual. The stress level is determined using a membership function, established using fuzzy logic.

### PRIOR ART

It is possible to determine a stress level of an individual on the basis of measurements of one or more physiological parameters of said individual. The physiological parameter may be a cardiac activity, for example measured via an electrocardiogram (ECG) or a simple determination of a cardiac frequency, or a muscular activity, measured via an electromyogram (EMG), or even a measurement of the electrical conductance of the skin. The publication Ollander “A comparison of wearable and stationary sensors for stress detection”, 2016 IEEE international conference on Systems, Man and Cybernetics (SMC), October 2016, describes how certain physiological parameters may be used to establish a stress indicator for an individual.

The emergence of portable connected sensors intended to be worn by an individual has made it possible to take measurements of physiological parameters both simply and inexpensively. It is for example a question of specific sensors able to be fastened to a bracelet or integrated into watches or connected to smart phones. For example, the device Empatica E4 comprises various sensors allowing physiological parameters such as electrodermal activity, cardiac activity or temperature to be easily accessed.

On the basis of the measured parameters, classification algorithms may be implemented, so as to determine whether the individual is in a stressed state or in a rest state. Certain classification algorithms are based on fuzzy logic. This type of algorithm is for example described in the publication Kumar M “Fuzzy techniques for subjective workload-score modeling under uncertainties”, IEEE transactions on systems, man and cybernetics—part B, Vol. 38, No. 6, December 2008. This type of algorithm requires a learning phase to be carried out, in which an individual is placed in a stressful situation, or in various stressful situations. The fact that a learning period, in which the individual is placed in a stressful situation, is required is constraining. In addition, the reliability of such methods may be compromised by physiological variability from one individual to the next.

The inventors propose a method for determining a stress level of an individual that does not require the individual to be placed in a stressed state during calibration. This allows the method to be implemented in a particularly simple manner, using equipment worn by the individual. In addition, the calibration may be repeated periodically.

### SUMMARY OF THE INVENTION

A first subject of the invention is a method for determining a stress level of an individual, depending on a physiological parameter of the individual, the value of which is liable to vary depending on the stress level of the individual, the method comprising the following steps:
a) measuring a physiological-parameter value in various calibration periods;
b) defining a range of variation in the physiological-parameter values measured in the various calibration periods;
c) depending on the range of variation, establishing a membership function, defining a stress level of the individual depending on the physiological-parameter value, the stress level defined by the membership function varying or being able to vary between:
   a rest level, corresponding to a rest state of the individual, the rest state preferably corresponding to the state in which the individual is in the calibration periods;
   a stressed level, corresponding to a stressed state of the individual;
   and at least one intermediate level, comprised between the rest level and the stressed level;
d) following c), measuring a physiological-parameter value during a measurement period, and determining the stress level of the individual, during the measurement period, by applying the membership function to the physiological-parameter value measured during the measurement period.

The method may be such that
in step c), the stressed level and each intermediate level are determined depending on the physiological-parameter values measured in the calibration periods, the individual preferably being in a rest state.
in step c), the stressed level and each intermediate level are determined depending on a distance between the physiological-parameter value measured in step d), i.e. in the measurement period, and the range of variation defined in step b);
in step a), in each calibration period, the individual is in a rest state or considered to be in a rest state.

According to an embodiment, c) comprises taking into account a threshold distance, such that
the rest level corresponds to a physiological-parameter value comprised in the range of variation resulting from b);
the stressed level corresponds to a physiological-parameter value a distance of which, with respect to the range of variation, is larger than the threshold distance;
each intermediate level corresponds to a physiological-parameter value outside of the range of variation, and the distance of which, with respect to the range of variation, is smaller than the threshold distance.

According to an embodiment, c) comprises calculating an dispersion indicator of the physiological-parameter values measured in the various calibration periods, the threshold distance been determined depending on the dispersion indicator. The dispersion indicator may be or may comprise an extent of the range of variation, corresponding to a deviation between a minimum value and a maximum value of the range of variation. The threshold distance may be obtained by applying a scale factor to the extent of the range of variation.

c) may comprise attributing a value representative of the range of variation, according to which:
the stressed state corresponds to a physiological-parameter value the distance of which, with respect to the representative value, is larger than the threshold distance;
each intermediate level corresponds to a physiological-parameter value the distance of which, with respect to the representative value, is smaller than the threshold distance.

According to an embodiment, the physiological parameter is or comprises:
a feature of the cardiac activity of the individual;
and/or a feature of a muscular activity of the individual;
and/or a feature of the cortical activity of the individual;
and/or a feature of the electrodermal activity of the individual;
and/or a body temperature of the individual;
and/or a feature representative of a movement of the individual.

According to an embodiment:
a) to c) are implemented independently of one another, respectively for various psychological parameters, in order to establish as many membership functions as psychological parameters in question, each membership function being associated with one physiological parameter;
d) comprises measuring various physiological parameters, during the measurement period, and determining a stress level of the individual relative to each physiological parameter, using each membership function respectively associated with each physiological parameter;

the method further comprising:

e) combining the stress levels determined relative to each physiological parameter, in d), in order to obtain a multi-feature stress-level index.

The combination may be or comprise a sum, or a weighted sum. In step e), the multi-feature stress-level index may be determined by calculating a weighted mean or a median of the stress levels respectively determined relative to each physiological parameter.

In this embodiment, in step c), each membership function may be defined independently of the others.

In this embodiment, with each physiological parameter may be associated a threshold distance and a range of variation of the physiological-parameter values measured during the calibration periods.

In one embodiment, the membership function, or each membership function, is a function defined in an interval comprised between the range of variation and the range of variation increased by the threshold distance. It may be continuous in this interval.

Another subject of the invention is a device for determining a stress level of an individual, comprising:
a sensor configured to measure a physiological parameter of the individual, the sensor measuring a parameter value liable to vary depending on the stress level of the individual;
a processor, configured to implement b) to c) of a method according to the first subject of the invention, on the basis of measurements of various physiological-parameter values measured by the sensor in calibration periods, the processor also being configured to implement d) on the basis of at least one physiological-parameter value measured during a measurement period.

Other advantages and features will become more clearly apparent from the following description of particular embodiments of the invention, which are given by way of nonlimiting example, and shown in the figures listed below.

### FIGURES

FIG. 1 shows a device allowing the invention to be implemented.

FIG. 2 illustrates the main steps of one embodiment of the invention.

FIGS. 3A, 3B and 3C show various examples of a membership function.

FIG. 4 illustrates the main steps of another embodiment of the invention.

FIGS. 5A and 5B show the results of experiments in which a multi-feature stress indicator was determined by implementing the invention.

### DESCRIPTION OF PARTICULAR EMBODIMENTS

FIG. 1 shows a device 1 allowing the invention to be implemented. A sensor 2 is placed against the body of an individual, for example on his wrist. The sensor 2 is configured to measure the value x(t) of a physiological parameter of the individual during a measurement period t. The term period designates a time interval, for example a few seconds or a few minutes. The value of the physiological parameter in question may vary depending on a stress level of the individual. The physiological parameter x may be a parameter such as described in the publications cited with respect to the prior art, in which it is designated by the term “feature”. The physiological parameter may for example be:
A parameter related to a cardiac activity of the individual, for example cardiac frequency. Such a parameter may be measured by electrodes similar to the electrodes of an electrocardiogram (ECG), or by optical means, of the photoplethysmography (PPG) type.
It may also be a question of a mean of the cardiac frequency or of a dispersion parameter of the cardiac frequency such as its standard deviation, calculated during a preset time period. The physiological parameter may result from a frequency analysis of the cardiac activity such as an analysis of the inter-beat interval (IBI). In an ECG, this interval may lie between two R peaks of two consecutive cardiac pulses. The frequency analysis may be carried out in frequency ranges comprised between 0 Hz and 0.5 Hz, and more specifically, for example between 0 and 0.003 Hz (ultra-low frequencies), or between 0.003 and 0.04 Hz (very low frequencies), between 0.04 Hz and 0.15 Hz (low frequencies), or between 0.15 Hz and 0.5 Hz (high frequencies).
A parameter related to a muscular activity of the individual, this type of parameter being able to be measured by electromyography (EMG).
A parameter related to the electrodermal activity of the individual, representative of electrical-conduction or electrical-impedance properties of the skin (for example electrodermal impedance or conductance) and/or capacitive properties of the skin. It may be a question of a mean value of the conductance of the skin or of the mean absolute value of a derivative of the conductance of the skin. It may also be a question of a frequency, or of a mean duration or of a mean amplitude of electrodermal responses calculated during a preset time period. More generally, the parameter in question is a feature of an electrodermal response.
A skin temperature of the individual.
A measurement of a movement, for example an acceleration along at least one axis, such a measurement allowing a tremble or a physical activity liable to impact the physiology of the individual to be measured. The parameter in question may be a norm of the acceleration.
A measurement of a cortical activity, for example taken using electroencephalography (EEG) electrodes. It may for example be a question of a spectral power in a preset frequency band, calculated in a preset time period.

The physiological parameter may be a parameter representing the cardiac frequency (or heart rate). If HRⱼ is the heart rate measured at an instant j, the physiological parameter x(t) at t may be:

$$x ( t ) = \sqrt{\frac{1}{{N}_{j}} {\sum }_{j} {( {HR}_{j} - {HR}_{j - 1} )}^{2}}$$

where Nⱼ is a number of heart reat measurements taken into account, and t−Nⱼ≤j≤t. The number Nⱼ is set so as to include the measurements of the hear rate during a sliding duration of a few seconds or a few tens of seconds, of a few minutes, for example 60 seconds.

The physiological parameter may be a parameter representing the inter-beat interval. If IBIⱼ is the inter-beat interval measured at an instant j, the physiological parameter x(t) at t may be:

$$x ( t ) = \sqrt{\frac{1}{{N}_{j}} {\sum }_{j} {( {IBI}_{j} - {IBI}_{j - 1} )}^{2}}$$

where Nⱼ is a number of inter-beat intervals taken into account, and t−Nⱼ≤j≤t. The number Nⱼ is set so as to include the measurements of the inter-beat interval during a sliding duration of a few seconds or a few tens of seconds, of a few minutes, for example 60 seconds.

The physiological parameters described in the two preceding paragraphs are to be considered as preferred parameters.

The objective of the invention is to determine a stress level Sl(t) of the individual in various measurement periods t.

The sensor 2 is connected to a microprocessor 4, the latter being connected to a memory 5 in which are stored instructions for implementing the method described above. The microprocessor 4 receives the measurements of the sensor 2, via a wired link or a wireless link. The microprocessor 4 may be worn/borne by the individual, being arranged with the sensor or being incorporated into an ancillary device carried by the person, for example a portable object such as a smart phone. The microprocessor 4 may also be remote from the individual.

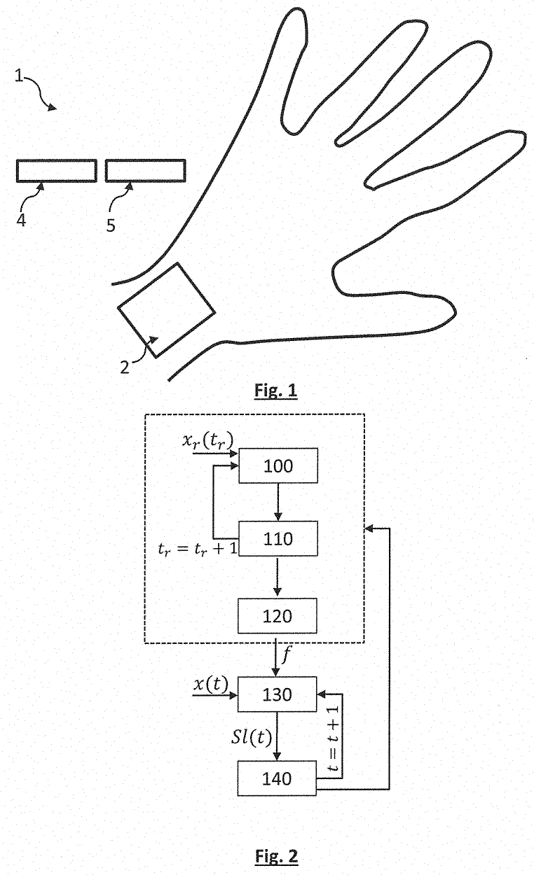

FIG. 2 describes the main steps of a first embodiment of the invention. The method requires a calibration phase, or learning phase, in which the method is parameterized. It is a question of establishing a membership function ƒ, which allows the measured parameter value to be related to a stress level. More precisely, and as described below, the membership function ƒ allows the measured parameter value to be related to a level of membership to a stressed state or to a rest state.

The calibration phase comprises steps 100 to 120. An important aspect of the invention is that in this phase, the individual is at rest, or, more precisely, considers himself as being at rest. Thus, the calibration phase comprises calibration periods tᵣ, in which periods the individual is considered as being solely in a rest state. He is therefore not in a stressed state. In the calibration phase, the sensor 2 measures a physiological-parameter value xᵣ(tᵣ), in various periods tᵣ, the index r designating the fact that the individual is considered as being at rest. If the calibration is carried out while the individual is in a certain stressed state, this degrades the reliability of the determination of the stressed state of the individual at measurement times subsequent to the calibration.

By rest state of an individual, what is for example meant is a state in which the individual is awake, but his physical and mental activity is minimal. For example, the individual is alone, sat or lying down, and performing no particular activity. In the rest of the description, the rest state corresponds to the state in which the individual is during the calibration.

In the measuring step 100, the sensor measures the physiological-parameter value xᵣ(tᵣ) corresponding to the calibration period tᵣ.

In step 110, the calibration period tᵣ is incremented, then step 100 is reiterated or the iteration loop formed by steps 100 and 110 is exited. In the step 110, the iteration loop may be exited at the end of a preset number of iterations, or depending on the values xᵣ(tᵣ) measured in the various calibration periods tᵣ. For example, it is possible to calculate a statistical quantity from the values xᵣ(tᵣ) measured in the calibration periods t, and to stop the iterations depending on a variation in this statistical quantity, and in particular when the variation in the statistical quantity is negligible. The statistical quantity may be the mean, or the median, or a dispersion indicator such as variance, standard deviation, or a deviation between a maximum value x_{r,max} and a minimum value x_{r,min} of the physiological parameter x.

In step 120, the values xᵣ(tᵣ) measured during the various calibration periods tᵣ are used to define the membership function ƒ. For example, a range of variation Xᵣ is defined in which the values xᵣ(tᵣ) measured during the various calibration periods tᵣ, which are called calibration values, lie. The range of variation Xᵣ is bounded by a minimum value x_{r,min} and a maximum value x_{r,max}. Thus, Xᵣ=[x_{r,min},x_{r,max}]. The range of variation may be characterized by its extent Δxᵣ. The extent is such that Δxᵣ=x_{r,max}−x_{r,min} (1).

Step 120 may comprise applying a statistical test in order to eliminate aberrant values xᵣ(tᵣ). A Dixon test, known to those skilled in the art, may for example be performed. The elimination of aberrant values allows the reliability of the method to be improved.

It is also possible to determine a threshold distance d_{S}. The threshold distance d_{S} may be such that:

d_{S}=α×Δxᵣ, (2), α being a real positive number, designated by the term scale factor. The value of the scale factor α depends on the physiological parameter in question. It is typically comprised between 0.1 and 0.5. The scale factor allows the threshold distance d_{S} to be determined from the scope Δxᵣ, as described below.

The membership function ƒ is intended to define a stress level Sl on the basis of a physiological-parameter value x(t) measured in a measurement period t, subsequent to the calibration phase. The stress level Sl may for example vary between 0 and 1, 0 corresponding to a rest state and 1 corresponding to a stressed state of the individual. According to the principles of fuzzy logic, the membership function ƒ may define intermediate levels, comprised between 0 and 1, and corresponding to an intermediate stressed state. The membership function ƒ is preferably continuous in an start space E defined by the values that the measured physiological parameter is capable of taking. The start space E may for example be the set of real positive numbers. Thus, ƒ: E= [symbol] ⁺→[0,1] and ƒ(x(t))=Sl(t).

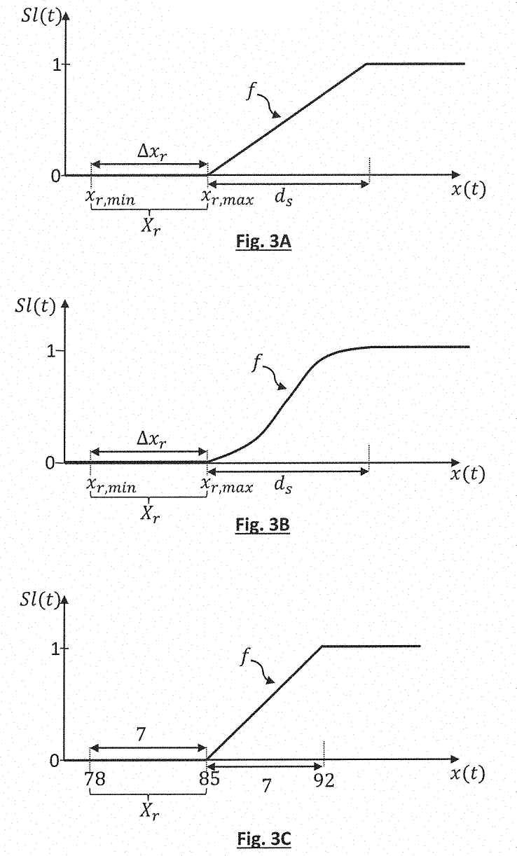

An example of a membership function ƒ is illustrated in the FIG. 3A:
When x(t)∈Xᵣ, i.e. when x_{r,min}≤x(t)≤x_{r,max}, Sl(t)=ƒ(x(t))=0 (3). In this case, the measured-parameter value x(t) belongs to the range of variation Xᵣ. The stress level of the individual is equal to 0, this meaning that the individual is considered as being at rest, i.e. as being in the same state as he was in during the calibration. The same also goes when x(t)≤x_{r,min}.
When x(t)≥x_{r,max}+d_{S}, Sl(t)=ƒ(x(t))=1 (4). The stress level of the individual is equal to 1, this meaning that the individual is considered as being in a stressed state. Thus, when the measured value x(t) of the parameter is distant by a distance larger than the threshold distance d_{S} from the range of variation Xᵣ, the individual is considered as being in a stressed state.
More generally, the membership function ƒ is such that when a distance d(x(t),Xᵣ) between the physiological-parameter value x(t) and the range of variation Xᵣ is larger than the threshold distance d_{S}, the stress level Sl(t) is equal to 1. In this example, d(x(t),Xᵣ)=d(x(t),x_{r,max}). Using the definition of threshold distance given in expression (2), condition (4) may be written:
If d(x(t),Xᵣ)>α×Δxᵣ, . . . , Sl(t)=ƒ(x(t))=1 (5).
The distance d(x(t), Xᵣ) may be a Euclidean distance or another distance.
When x_{r,max}<x(t)<x_{r,max}+d_{S}, Sl(t)=ƒ(x(t))∈]0,1[(6). The stress level of the individual is an intermediate level, comprised between 0 and 1. In the example shown in FIG. 3A, the membership function is piecewise linear. Thus, in this example, when x_{r,max}≤x(t)≤x_{r,max}+d_{S},

$$Sl ( t ) = f ( x ( t ) ) = \frac{x ( t ) - {x}_{r , max}}{{d}_{S}} .$$

The normalisation by d_{S} allows intermediate state levels comprised between 0 (when x(t)=x_{r,max}) and 1 (when x(t)=x_{r,max}+d_{S}) to be obtained. Thus, when the measured value is comprised between x_{r,max} and x_{r,max}+d_{S}, the value of the membership function varies monotonically between 0 and 1. In this example, the membership function is an increasing function. The closer x(t) gets to x_{r,max}+d_{S}, the higher the value of the membership function.
More generally, the membership function ƒ is such that: when x(t)∉Xᵣ and d(x(t),Xᵣ)<d_{S}, (7) the stress level is an intermediate level, comprised between the level corresponding to the rest state (i.e. the state in which the calibration was carried out) and the level corresponding to the stressed state. When the rest and stressed levels are set to 0 in 1, respectively, and the membership function is linear,

$$Sl ( t ) = \frac{d ( x ( t ) , {X}_{r} )}{{d}_{S}} . \qquad ( 8 )$$

According to one variant, shown in FIG. 3B, the function ƒ is not piecewise linear. It may for example take the form of a hyperbolic tangent or any other sigmoid function.

The example given above is valid when the membership function ƒ is an increasing function, i.e. when the stress level increases as the measured value of the physiological parameter increases. In certain particular cases, for example when the parameter in question is skin resistance, the membership function ƒ is a decreasing function: as the stress level increases, the measured parameter value decreases. In such a configuration:
when x_{r,min}≤x(t), Sl(t)=ƒ(x(t))=0;
when x(t)≤x_{r,min}−d_{S}, Sl(t)=ƒ(x(t))=1 (9);
when x_{r,min}−d_{S}<x(t)<x_{r,min}, Sl(t)=ƒ(x(t))∈]0,1[(10). If the membership function is piecewise linear, then when x_{r,min}−d_{S}<x(t)<x_{r,min},

$$Sl ( t ) = f ( x ( t ) ) = \frac{{x}_{r , min} - x ( t )}{{d}_{S}} .$$

The normalization by d_{S} allows intermediate state levels comprised between O (x(t)→x_{r,min}) and 1 (x(t)→x_{r,min}−d_{S}) to be obtained. The arrow → means “tends toward”.

Generally, it is possible to attribute a representative value to the range of variation Xᵣ. Depending on the distance d between the parameter value x(t) and the representative value, the membership function ƒ determines a stress level Sl(t). In the examples given above, the representative value was respectively set equal to the maximum value x_{r,max} and to the minimum value x_{r,min} of the range of variation. In other examples, the representative value may be a statistical indicator applied to the calibration values xᵣ(tᵣ) measured during the calibration. It may for example be a question of the mean Xᵣ or of the median med(Xᵣ) of the calibration values. It may also be a question of a fractile, for example a quartile (the first quartile, when the membership function is a decreasing function or the fourth quartile when the membership function is an increasing function) or a decile (for example the first decile when the membership function is a decreasing function or the tenth decile when the membership function is an increasing function). The stress level, corresponding to a measured parameter value x(t), may then be calculated depending on the distance between the value of the parameter and the value representative of the range of variation. The distance d may be normalised by an indicator of the dispersion of the calibration values, for example the extent Δxᵣ of the range of variation or the standard deviation of the calibration values xᵣ(tᵣ).

The scale factor α may be determined depending on an indicator of the dispersion of the values measured during the calibration. The dispersion indicator may be the extent Δxᵣ of the range of variation Xᵣ. It may also be a question of a variance or a standard deviation of the calibration values xᵣ(tᵣ).

Steps 130 and 140, which are described below, correspond to a phase of use of the sensor 2 to estimate a stressed state of the individual for whom the membership function ƒ was defined. In step 130, a measurement of the physiological parameter x(t) is carried out in a measurement period t. The physiological parameter x(t) measured in each measurement period is the same as that measured in the calibration periods.

In step 140, the membership function ƒ defined beforehand is applied to the value x(t) of the physiological parameter in the measurement period, so as to determine a stress level Sl(t)=ƒ(x(t)). At the end of step 140, the period may be incremented and another iteration of steps 130 to 140 carried out.

Thus, in the method described above, the calibration is carried out solely with parameter values measured in the calibration, while the individual is considered as being in a rest state. The calibration does not require parameter measurements to be carried out while the individual is in a stressed state. One advantage of the method is that the calibration is faster and simpler to carry out. Another advantage is that the calibration may be repeated periodically, in order to take into account a possible physiological variability of the user. In this case, when a repetition is desired, following step 140, the method implement steps 100 to 120. Since the calibration is particularly simple to carry out, it is possible to frequently repeat the calibration.

One example application is shown in FIG. 3C, the measured parameter being a mean cardiac frequency during a period of one minute. In the learning phase, the measured values of the mean cardiac frequency were between 78 and 85 beats per minute. Thus, Δxᵣ=7. The membership function such as shown in FIG. 3C is then obtained by considering α=1. Thus:
when x(t)<85, the stress level is equal to 0, the individual being in a rest state;
when 85<x(t)≤92, the stress level is comprised between 0 and 1;
when x(t)>92, the stress level is equal to 1, the individual being in a stressed state.

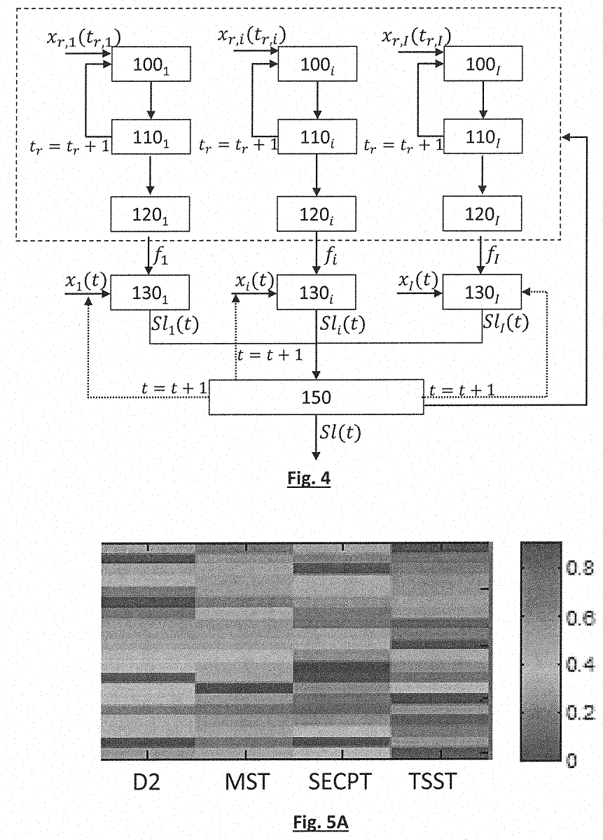

According to one embodiment, the method described above may be applied while simultaneously measuring various parameters xᵢ, the index i identifying the parameter in question, with 1<i≤I, I designating the number of physiological parameters in question. FIG. 4 shows the main steps of this embodiment.

For each physiological parameter xᵢ, a calibration is carried out in steps 100ᵢ, 110ᵢ and 120ᵢ. These steps are respectively similar to steps 100, 110 and 120 described above. They are respectively implemented on the basis of values x_{r,1}(tᵣ) . . . x_{r,i}(tᵣ) . . . x_{r,I}(tᵣ) of the physiological parameters in question, in different calibration periods tᵣ.

Each step 120ᵢ is carried out considering a range of variation X_{r,i}, of the parameter xᵢ during the calibration. The range of variation X_{r,i} has an extent Δx_{r,i}. To each physiological parameter xᵢ is assigned a scale factor αᵢ. It will be noted that the scale factor αᵢ may be different from one physiological parameter to the next. The step 120ᵢ allows a membership function ƒᵢ relative to the physiological parameter xᵢ to be defined. The membership functions ƒᵢ, ƒᵢ₊₁, respectively associated with two different parameters xᵢ,xᵢ₊₁, are established independently of each other. It is however preferable that, for each membership function, the rest state and the stressed state correspond respectively to the same levels, for example 0 for the rest state and 1 for the stressed state. Thus, a definition of I membership functions ƒ₁ . . . ƒ_{I}, respectively associated with the I measured physiological parameters x₁ . . . x_{I}, is achieved. To each physiological parameter in question may correspond one range of variation, determined in the calibration, and one threshold distance. Each membership function ƒᵢ is established depending on the range of variation and on the threshold distance that are associated with each physiological parameter.

After each membership function ƒᵢ has been defined, the method comprises a step 130ᵢ, implemented for each parameter xᵢ(t) measured in a measurement period t, so as to determine a stress level Slᵢ(t) associated with each parameter xᵢ(t), according to the expression Slᵢ(t)=ƒᵢ(xᵢ(t)). Thus, a definition of I stress levels Sl₁(t) . . . Sl_{I}(t), respectively associated with the I physiological parameters x₁ . . . x_{I} in question, is achieved.

In a step 150, the various stress levels Sl₁(t) . . . Sl_{I}(t), respectively associated with each parameter xᵢ(t), are combined, so as to determine an overall, or multi-feature, stress level Sl(t), according to the principles of fuzzy logic. The combination may be a calculation of a mean value or of a median value. It may also be a question of a weighted mean, in which each stress level Slit) is assigned a weighting factor λᵢ dependent on the importance that it is desired to attribute to the physiological parameter xᵢ relative to the other parameters in question. The various stress levels Sl₁(t) . . . Sl_{I}(t) may be combined by applying predetermined inference rules.

On the basis of the multi-feature stress level Sl(t), it is possible to determine whether the individual is in a stressed state. Initial experimental trials have shown that when the multi-feature stress level Sl(t) is higher than 0.3 or 0.4, the individual may be considered to be in a stressed state in the measurement period.

Moreover, on the basis of the multi-feature stress level Sl(t), it is possible to define an activity state, corresponding to an intermediate state between the rest state and the stressed state. The activity state corresponds to an individual performing a normal mental or physical activity, without being in a stressed state. The activity state corresponds to a multi-feature stress level lying between:
the multi-feature stress level for which the individual is considered to be in a stressed state;
and the multi-feature stress level for which the individual is considered to be in a rest state.

Experimental trials have been carried out on a cohort of 20 subjects aged from 19 to 30 years, who were successively subjected to four different stressful situations:
A first stressful situation, known in the art by the term “test D2”, in which the subject must recognise, in a limited time, symbols distributed among visually similar symbols, the latter being referred to as “distractors”. It is a question of a conventional selective attention test.
A second stressful situation, known in the art by the term “MST”, meaning “Mental Stress Test”, in which the subject must count, downward, starting (for example) from the number 1022, in increments of 13, while being filmed and in the presence of two observers.
A third stressful situation, known in the art by the term “SECPT”, meaning “Socially Evaluated Cold-Pressure Test”, in which the subject places his hand in very cold water (from 0° to 4° C.) for three minutes, in the presence of an observer.
A fourth stressful situation, known in the art by the term “TSST”, meaning “Trier Social Stress Test”, in which the subject is filmed during a simulated recruitment interview in front of two observers.

In each test, three types of physiological signals were measured:
cardiac activity was measured using two ECG electrodes;
cutaneous conductance was measured by virtue of two electrodes located on the middle phalanx of the middle finger and of the index finger of the non-dominant hand;
muscular activity was measured using EMG electrodes placed on the corrugator muscle of the eyebrow and of the upper and lower orbicularis oris muscles.

The signals were acquired as an acquisition frequency of 1000 Hz.

In a preliminary phase, the parameters x, or features, that were the most suitable for the detection of a stressful situation were selected. It was a question of:
the mean of the cardiac frequency;
the standard deviation of the cardiac frequency;
a mean spectral power of the inter-beat interval in the ultralow-frequency spectral band;
a mean spectral power of the inter-beat interval in the very-low-frequency spectral band;
the mean cutaneous conductance;
the mean of the absolute value of the derivative of the cutaneous conductance;
the proportion of positive samples in the derivative of the cutaneous conductance.

The value of each parameter was calculated for a measurement period comprised between 3 and 5 minutes.

For each parameter, a membership function was established, as described above.

Next, each parameter, and its membership function, were combined in order to form a multi-feature stress indicator. This index was obtained by calculating a mean of the value of the membership function for each parameter.

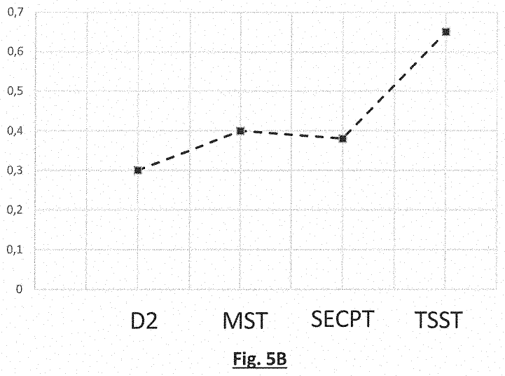

FIG. 5A shows, for each stressful situation (x-axis) and for each tested individual (y-axis), a value of the multi-feature stress indicator, which value is represented on a greyscale.

FIG. 5B shows the mean value, for all of the tested individuals, of the multi-feature stress indicator.

It may be seen that the highest values of the stress indicator are obtained for the TSST test.

The invention will possibly be employed to track the stress level of individuals. It may for example be a question of tracking stress level in a professional environment, or of tracking the stress level of individuals who are subject to anxiousness in particular situations, for example in a means of transportation. It may also be applied to track the stress level of an athlete.

1. A Method for determining a stress level of an individual, depending on a physiological parameter of the individual, the value of which is liable to vary depending on the stress level of the individual, the method comprising:
a) measuring a physiological-parameter value, in various calibration periods;
b) defining a range of variation in the physiological-parameter values measured in the various calibration periods;
c) depending on the range of variation, establishing a membership function, the membership function defining a stress level of the individual depending on the physiological-parameter value, the stress level defined by the membership function varying between:
a rest level, corresponding to a rest state of the individual;
a stressed level, corresponding to a stressed state of the individual;
and at least one intermediate level, comprised between the rest level and the stressed level;
d) following c), measuring a physiological-parameter value in a measurement period, and determining a stress level of the individual, during the measurement period, by applying the membership function to the physiological-parameter value measured during the measurement period;
wherein in c), the stressed level and each intermediate level are determined:
depending on the physiological-parameter values measured in the calibration periods;
and depending on a distance between the physiological-parameter value measured in d) and the range of variation defined in b);
and wherein, in a), in each calibration period, the individual is considered to be in a rest state.

2. The Method according to claim 1, wherein c) comprises taking into account a threshold distance, such that:
the rest level corresponds to a physiological-parameter value comprised in the range of variation resulting from b);
the stressed level corresponds to a physiological-parameter value a distance of which, with respect to the range of variation, is larger than the threshold distance;
each intermediate level corresponds to a physiological-parameter value outside of the range of variation, and the distance of which, with respect to the range of variation, is smaller than the threshold distance.

3. The method according to claim 2, wherein c) comprises calculating an dispersion indicator of the physiological-parameter values measured in the various calibration periods, the threshold distance been determined depending on the dispersion indicator.

4. The method according to claim 3, wherein the dispersion indicator is or comprises an extent of the range of variation, corresponding to a deviation between a minimum value and a maximum value of the range of variation.

5. The method according to claim 4, wherein the threshold distance is obtained by applying a scale factor to the extent of the range of variation.

6. The method according to claim 2, wherein c) comprises attributing a value representative of the range of variation, according to which:
the stressed state corresponds to a physiological-parameter value the distance of which, with respect to the representative value, is larger than the threshold distance;
each intermediate level corresponds to a physiological-parameter value the distance of which, with respect to the representative value, is smaller than the threshold distance.

7. The Method according to claim 1, wherein the physiological parameter is or comprises:
a feature of the cardiac activity of the individual;
and/or a feature of a muscular activity of the individual;
and/or a feature of the cortical activity of the individual;
and/or a feature of the electrodermal activity of the individual;
and/or a body temperature of the individual;
and/or a feature representative of a movement of the individual.

8. The Method according to claim 1, wherein:
a) to c) are implemented independently of one another, respectively for various psychological parameters, in order to establish as many membership functions as psychological parameters in question, each membership function being associated with one physiological parameter;
d) comprises measuring various physiological parameters, during the measurement period, and determining a stress level of the individual relative to each physiological parameter, using each membership function respectively associated with each physiological parameter;
the method comprising:
e) combining the stress levels determined relative to each physiological parameter, in d), in order to obtain a multi-feature stress-level index.

9. The method according to claim 8, wherein, in e), the multi-feature stress-level index is determined by calculating a weighted mean or a median of the stress levels respectively determined relative to each physiological parameter.

10. The method according to claim 8, wherein, in c), each membership function is defined independently of the others.

11. The method according to claim 2, wherein the membership function, or each membership function, is a function defined in an interval comprised between the range of variation and the range of variation increased by the threshold distance, and is continuous in this interval.

12. A device for determining a stress level of an individual, comprising:
a sensor configured to measure a physiological parameter of the individual, the sensor measuring a parameter value liable to vary depending on the stress level of the individual;
a processor, configured to implement b) to c) of the method according to claim 1, on the basis of measurements of various physiological-parameter values measured by the sensor in calibration periods, the processor also being configured to implement d) on the basis of at least one physiological-parameter value measured during a measurement period.
